(19) United States
(12) Patent Application Publication
(10) Pub. No.: US 2016/0067292 A1
(43) Pub. Date: Mar. 10, 2016

(54) BIOACTIVE APOLAR EXTRACT CONTAINING PLANT GENETIC MATERIAL FOR TREATMENT OF MAMMALIAN DISEASES

(71) Applicants: Munisekhar MEDASANI, Hyderabad (IN); Satyasayee Babu DIVI, Vishakapatnam (IN)

(72) Inventors: Munisekhar Medasani, Hyderabad (IN); Satyasayee Babu Divi, Vishakapatnam (IN)

(21) Appl. No.: 14/783,133

(22) PCT Filed: Aug. 4, 2014
(86) PCT No.: PCT/IN2014/000223

§ 371 Date: Oct. 8, 2015

(30) Foreign Application Priority Data
Apr. 8, 2013 (IN) 1598/CHE/2013

(51) Int. Cl. A61K 36/185; C12N 15/113

(52) U.S. Cl. CPC A61K 36/185; C12N 15/113; C12N 2310/11; C12N 2320/30

(57) ABSTRACT

Bioactive apolar extract of plant material containing plant genetic material to treat mammalian diseases and disorders.

### BACKGROUND OF INVENTION

MicroRNAs (miRNAs) are small, endogenous RNAs that regulate gene expression in plants and animals. In plants, these ˜21-nucleotide RNAs are processed from stem-loop regions of long primary transcripts by a Dicer-like enzyme and are loaded into silencing complexes, where they generally direct cleavage of complementary mRNAs. (1)

miRNAs are deeply conserved within both the plant and animal kingdoms, there are substantial differences between the two lineages with regard to the mechanism and scope of miRNA-mediated gene regulation; several of these differences have been instrumental in the rapid increase in understanding of plant miRNA biology. Plant miRNAs are highly complementary to conserved target mRNAs, which allows fast and confident bioinformatic identification of plant miRNA targets (1)

As understanding of miRNA genomics and function in plants and animals has grown, so has the realization that there are numerous differences between the kingdoms in the ways miRNAs are generated and carry out their regulatory roles. Indeed, the evolutionary relationship between plant and animal miRNAs is unclear.

(1) Cellular miRNAs play fundamental roles during basic cellular processes such as differentiation, development and death. In addition, studies involving miRNAs and their dysregulation have helped identify key concepts in infectious diseases, cancer and both generation and maintenance of adequate innate and adaptive immune responses.

(2) The cellular context of HIV-1 infection is one of the primary determinants of a successful outcome, and the cell type-specific expression or activity of miRNAs must be considered in the relevant environment. Individual miRNAs may be downregulated upon macrophage differentiation but remain unchanged or increased following CD4+ T cell activation, as is the case for miR-198. While miR-198 represses Cyclin T1 protein levels in monocytes, this function appears to be supplied by miR-27b, 29b, 150, and 223 in resting CD4+ T cells, suggesting that Cyclin T1 suppression is important in both cell types, although each has employed different miRNA expression patterns to achieve the same goal.

(3) miRNAs act in a complex functional network in which each miRNA might control hundreds of distinct target genes, and the expression of a single coding gene can be regulated by many different miRNA. In line with this, recent evidence shows their important role in the regulation of a multitude of physiological functions, such as stem cell differentiation, neurogenesis, hematopoiesis, immune response, and skeletal and cardiac muscle development and stress. Furthermore, a variety of diseases, such as cancer, diabetes, and heart hypertrophy and failure, have been related to aberrant expression of miRNAs.

(4) Laurent Houzet et al has reported that HIV-1 infection generally resulted in the down regulation of most human miRNAs in vivo.

(5) Lin Zhang et al reported the surprising finding that exogenous plant miRNAs are present in the sera and tissues of various animals and that these exogenous plant miRNAs are primarily acquired orally, through food intake. (6) . Though their finding has been questioned later by other researchers, there was no confirmed evidence provided to show Lin Zhang et al finding was wrong.

(7) Hervé Vaucheret and Yves Chupeau have studied hypothesis that epithelial cells in the intestine might take up miRNAs in food, package them into MVs and release them into the circulatory sytem.

(8). But they have tested the hypothesis in invitro and could not prove it yet on humans.

Above literature review suggest that there is interaction between plant miRNA in regulating human processes. Genetic materials like microRNA and anti-sense microRNA from the plant kingdom could be playing role in human disease progression and control.

In the prior art it could not identify specific class of plants which have therapeutic value to mammals without harm to the healthy cells or organs. We have identified the class of plants which has therapeutic effect on the diseases in invitro and invivo and also observed that non-coding genetic material alongwith NADPH Oxidase inhibitors have shown good results in controlling and treating infectious diseases, tumours, cancers, hepatic diseases and hyperlipidemia. Also our process and test results proves that plants' non-coding genetic material passes through the digestive system and enters into the blood stream, organs and other parts of the body without getting denatured. This finding opens door for natural plant genetic material for treatment of diseases even if administered by oral route.

### DETAILED DESCRIPTION

The present invention is relating to the Bioactive apolar extract of plant material containing bioactive undenatured plant genetic material for treatment of mammalian diseases and disorders.

In our in-vitro and in-vivo research, both cancerous and non-cancerous, we observed that apolar extract of high altitude plants have inhibited progression of diseases where there is immune support (like in-vivo) and where there is no immune support (like in-vitro). This inhibitory effect is selective to the diseases and does not have any adverse effect on the normal, healthy cell lines. We believe that this effect is achieved by the Plant RNA material, mainly non-coding small RNAs like miRNA and anti-sense miRNA. Inhibition of cancer cell lines and DNA virus, non-toxicity towards normal healthy cell lines and in-vitro effect (without in-vivo immunity support) is eluding to the possibility that the effect of plant miRNA or antisense miRNA. This plant RNA material along with other bioactive substances of plant extract is expected to have beneficial effect in treating mammalian diseases and disorders.

Inventors have observed that these extracts containing plant genetic materials, mainly plant miRNA or antisense miRNA along with other plant bioactive substances like NADPH Oxidase inhibitors have shown control and reduction of neurological diseases, liver diseases, hyperlipidemia, inflammation, tumours, cancers, viral infections, retroviral diseases, proviral control, bacterial, fungal and parasitic infections.

Inventors have also observed that apolar plant extract from underground parts of high altitude plants containing miRNA and NADPH Oxidase inhibitors have controlled brain tumours with oral ingestion and lead to good prognostic outcome. They have also observed in invivo studies that these have improved the functionality of the liver, have a positive effect on atherosclerosis and lipid profile. In invivo studies, it was also observed that it has reduced ROS in the brain area and has improved overall cognitive function.

Inventors also have identified many other plants and its parts containing plant genetic materials like small RNAs, specially microRNAs and anti-sense microRNAs have wide preventive and treatment effect on the diseases like neurological, neuromuscular, lipid disorders, hyperlipidemia, hepatic diseases and disorder, tumours, cancers, infectious diseases including viral, bacterial and parasitic, retroviral diseases.

Inventors also working on other human sufferings like autoimmune hair fall, obesity, pigmentation, metabolic syndromes etc. Preliminary results indicate that hyper pigmentation can be controlled by exogenous micro RNA from the plant material. Inventors' also anticipating plant extract prepared without denaturing the small RNAs mainly microRNAs and anti-sense microRNAs can provide prevention and treatment to the most of mammalian diseases and disorders.

Inventors have observed that apolar solvents are preferred solvent media for plant small RNA and other bioactive components extraction. It is more preferred to use acidic apolar solvents than base apolar solvents.

Invivo tests with plant extract containing microRNAs and NADPH Oxidase inhibitors show that these small non-coding RNAs like miRNAs and anti-sense miRNAs along with other bioactive compounds from the same or other plants can provide prevention or treatment to the mammalian diseases and disorders. These bioactive compounds could be enzymes, proteins, lipids, carbohydrates and others which are natural found in nature and plant kingdom. Many of these bioactive compounds in nature are available with very weak bonds like oxygen or nitrogen or phosphorus. For example, glycoproteins are proteins attached to the sugar moiety. When this gets into digestive system by food intake, weak O bond will be hydrolysed and proteins will be released into body system for its action. Many of these bioactive compounds have to be reduced to active form, for increased efficacy. Our process will address this hydrolysis outside the body environment and provides only bioactive compounds to the body which are active and more effective.

It is anticipated and disclosed here that any other studies done in the future which are within the scope of this description and scope of this patent forms part of this invention.

### Embodiment

Following are the working examples for preparation of apolar plant extract. Any other working examples generated by persons skilled in the art, within the scope of this invention, forms part of the patent.

### Example #1

Cleaning, Washing, Drying of aerial parts of Hibiscus plant at below 10 deg C. temperature till the plant material is dry enough to pulverize.
Pulverizing the plant material to coarse powder at 20 deg C. temperature, under dry nitrogen gas atmosphere
Extraction with activated naphtha solvent at 20 deg C. temperature
Collecting liquid extract and evaporating the solvent at 20 deg C. temperature under dry nitrogen gas atmosphere
Drying apolar plant extract at 20deg C. temperature, under dry nitrogen gas atmosphere
Packing and formulating the dried apolar plant extract at 20 deg C. temperature, under dry nitrogen gas atmosphere

### Example #2

Cleaning, Washing, Drying of underground parts of high altitude plant at below 5 deg C. temperature till the plant material is dry enough to pulverize.
Pulverizing the plant material to coarse powder at 10 deg C. temperature, under dry nitrogen gas atmosphere
Extraction with acidic liquid CO2 as solvent.
Collecting liquid extract and evaporating it by changing the pressure at below 5 deg C. temperature
Drying apolar plant extract at 5 deg C. temperature, under dry argon gas atmosphere
Packing and formulating the dried apolar plant extract at 20 deg C. temperature, under dry argon gas atmosphere

### Example #3

Cleaning, Washing, Drying of see weed at below 10 deg C. temperature till the plant material is dry enough to pulverize.
Pulverizing the plant material to coarse powder at 20 deg C. temperature, under dry nitrogen gas atmosphere
Extraction with activated iso-hexane solvent at 20 deg C. temperature
Collecting liquid extract and evaporating the solvent at 10 deg C. temperature under dry nitrogen gas atmosphere
Drying apolar plant extract at 10 deg C. temperature, under dry nitrogen gas atmosphere
Packing and formulating the dried apolar plant extract at 10 deg C. temperature, under dry nitrogen gas atmosphere

While the invention has been described in relation to preferred embodiments of the invention, it will be appreciated that other embodiments, adaptations and modifications of the invention will be apparent to those skilled in the art.

A person skilled in the art can easily produce any number of preferred embodiments identified above. All such embodiments, variations, modifications forms part of the present invention.

### REFERENCES

1. Matthew W. Jones-Rhoades et al, MicroRNAs and Their Regulatory Roles in Plants, Annu. Rev. Plant. Biol. 2006.57:19-53.

2. Swaminathan Gokul, et al, MicroRNAs and HIV-1 Infection: Antiviral Activities and Beyond, J Mol Biol 2013

3. Karen Chiang and Andrew P. Rice, MicroRNA-Mediated Restriction of HIV-1 in Resting CD4+ T Cells and Monocytes, Viruses 2012, 4, 1390-1409

4. Catalucci et al, MicroRNAs in Cardiovascular Biology and Heart Disease, Circ Cardiovasc Genet 2009; 2; 402-408

5. Laurent Houzet et al, MicroRNA profile changes in human immunodeficiency virus type 1 (HIV-1) seropositive individuals, Retrovirology 2008, 5:118

6. Lin Zhang et al, Exogenous plant MIR168a specifically targets mammalian LDLRAP1: of cross-kingdom regulation by microRNA, Cell Research (2012) 22:107-126

7. Web Link: http://www.the-scientist.com/?articles.view/articleNo/31975/title/Plant-RNA-Paper-Questioned/

8. Hervé Vaucheret and Yves Chupeau, Ingested plant miRNAs regulate gene expression in animals, Cell Research (2012) 22:3-5.

1. Bioactive apolar extract of plant material containing bioactive undenatured plant genetic material for treatment of mammalian diseases and disorders.

2. Bioactive apolar extract of plant material according to claim 1, wherein the extract of plant material is derived from aerial, underwater parts of the plants from plant kingdom.

3. Bioactive apolar extract of plant material according to claim 1, wherein the plants material is from, but not limited to, high altitude plants which grow above 8,000 feet altitude.

4. Bioactive apolar extract of plant material according to claim 1, wherein the plants material is derived from, but not limited to, the underground parts.

5. Bioactive apolar extract of plant material according to claim 1, wherein the plant genetic material is non-coding small RNAs.

6. Bioactive apolar extract of plant material according to claim 1, wherein the plant genetic material is microRNAs.

7. Bioactive apolar extract of plant material according to claim 1, wherein the plant genetic material is antisense microRNAs.

8. Bioactive apolar extract of plant material according to claim 1, wherein the bioactive apolar plant extract further contains, but not limited to NadPH Oxidase inhibitors of the same or other plant material.

9. Bioactive apolar extract of plant material according to claim 1, wherein mammalian diseases and disorders are neurological, neurodegenerative, neuromuscular, oxidative stress, HIV, AIDS, Cancer, tumour, hyperlipidemia, hepatic diseases, hypothyroidism, autoimmune disorder, metabolic disorder, skin disorders.

10. Bioactive apolar extract of plant material according to claim 1, wherein bioactive apolar extract contains less than 20 percent of dipolar constituents and less than 2 percent of sugar moieties.

11. Bioactive apolar extract of plant material according to claim 1, wherein extract of plant material is highly flammable with very low flash point.

12. Bioactive apolar extract of plant material according to claim 1, wherein the extract of plant material is in solid or semi-solid form at 20 deg C..

13. Bioactive apolar extract of plant material according to claim 1, wherein the extract of plant material is formulated into tablet, caplet, capsule, gel cap, injection, syrup, patch for administration to mammalians.

14. Bioactive apolar extract of plant material according to claim 13, wherein method of administration is oral, transdermal, subcutaneous, IV, IM in medically accepted dosage.

15. Bioactive apolar extract of plant material according to claim 13, wherein administration dosage is from 10 mg to 1,000 mg per day in dry form.

16. Bioactive apolar extract of plant material according to claim 13, wherein formulation is further added with adjuvants to enhance the taste, flavour, appearance, colour, stability, efficacy.

17. Bioactive apolar extract of plant material according to claim 13, wherein formulation is administered to mammalians as food supplement or prophylactic agent or medicine.

18. Bioactive apolar extract of plant material from aerial and/or underground and/or underwater parts of plants containing undenatured genetic and other bioactive material has been obtained by:
Cleaning, Washing, Drying of parts of plants at below 10 deg C. temperature
Pulverizing the plant material to coarse powder at below 20 deg C. temperature, under inert gas atmosphere.
Extraction with activated apolar solvent at below 20 deg C. temperature.
Collecting liquid extract and evaporating the solvent below 20 deg C. temperature under inert gas atmosphere.
Drying apolar plant extract below 30 deg C. temperature, under inert gas atmosphere.
Packing and formulating the dried apolar plant extract below 20 deg C. temperature, under inert gas atmosphere.
